(19) United States
(12) Patent Application Publication
(10) Pub. No.: US 2023/0128191 A1
(43) Pub. Date: Apr. 27, 2023

(54) METHODS AND KITS FOR THE DETECTION OF SARS-COV-2

(71) Applicant: THE TRANSLATIONAL GENOMICS RESEARCH INSTITUTE, Phoenix, AZ (US)

(72) Inventors: David Engelthaler, Flagstaff, AZ (US); Jolene Bowers, Flagstaff, AZ (US); James Schupp, Flagstaff, AZ (US)

(21) Appl. No.: 17/911,350

(22) PCT Filed: Mar. 12, 2021
(86) PCT No.: PCT/US21/22132

§ 371 Date: Sep. 13, 2022

Related U.S. Application Data
(60) Provisional application No. 62/989,554, filed on Mar. 13, 2020.
(60) Provisional application No. 62/989,550, filed on Mar. 13, 2020.
(60) Provisional application No. 63/076,811, filed on Sep. 10, 2020.

(51) Int. Cl. C12Q 1/70

(52) U.S. Cl. CPC C12Q 1/701; C12Q 1/6851

(57) ABSTRACT

Methods, kits, and oligonucleotides used in the detection of the coronavirus strain, severe acute respiratory syndrome coronavirus 2 (SARS-CoV-2), are disclosed. In some aspects, the oligonucleotides are primers or probes used in the described methods or kits. The oligonucleotide consists of 42 or less nucleotides and has a nucleotide sequence that consists essentially of, or is a variant of, the nucleotide sequence of: SEQ ID NO: 1, SEQ ID NO:2, SEQ ID NO:3, SEQ ID NO:5, SEQ ID NO:6, or SEQ ID NO:7. In some embodiments, the oligonucleotide is modified with an internal spacer or a detectable label. For example, the 5′ terminus is labeled with a fluorophore and the 3′ terminus is complexed to a quencher of fluorescence of said fluorophore. In some embodiments, the nucleotide sequence of the oligonucleotide further comprises a universal tail sequence.

### CROSS REFERENCE TO RELATED APPLICATIONS

This application claims the benefit of U.S. provisional patent application No. 62/989,550, filed Mar. 13, 2020, titled “Methods and Kits for the Detection of SARS-CoV-2;” U.S. provisional patent application No. 62/989,554, filed Mar. 13, 2020, titled “Methods and Kits for the Detection of SARS-CoV-2 and MERS-CoV;” and U.S. provisional patent application No. 63/076,811, filed Sep. 10, 2020, titled “Methods and Kits for the Detection of SARS-CoV-2,” the entirety of the disclosures of which are hereby incorporated by this reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 3,020 byte ASCII (text) file named “SeqList” created on Mar. 3, 2021.

### TECHNICAL FIELD

The present invention relates to the field of detection of coronavirus, and more particularly, severe acute respiratory syndrome coronavirus 2 (SARS-CoV-2), which has been implicated in the pathogenesis of the disease COVID-19.

### BACKGROUND

The world is experiencing a global outbreak of coronavirus disease COVID-19 caused by SARS-CoV-2, which was first reported in China in December 2019. Symptoms of COVID-19 is flu-like symptoms and can lead to pneumonia or more severe conditions. However, most people infected with the COVID-19 virus and develop symptoms will experience only mild to moderate respiratory illness and recover without requiring special treatment. Older people, and those with underlying medical problems like cardiovascular disease, diabetes, chronic respiratory disease, and cancer are more likely to develop serious illness. More than a year after the first reported case of COVID-19, there still remains no specific treatment for COVID-19.

Unlike most other respiratory disease, COVID-19 is known to spread even from an asymptomatic infected person to a close contact. An estimated 40% of individuals with SARS-CoV-2 infection are asymptomatic. Accordingly, SARS-CoV-2 can easily quietly spread within the community. Identifying where SARS-CoV-2 infections are taking place in the community is key to slowing the spread of COVID-19. Unfortunately, limitations in identifying the infection resulted in COVID-19 being declared a pandemic by the World Health Organization. To date, the pandemic has yet to end, and SARS-CoV-2 continues to place public health and economic stresses on the world. Identification of the etiology of COVID-19 and related illnesses is important in order to understand risk factors, target surveillance, properly treat diagnosed COVID-19 patients, and to help limit additional outbreaks. Thus, detecting SARS-CoV-2 infection as early and as fast as possible with a sensitive, reliable test remains crucial for ending the COVID-19 pandemic.

### SUMMARY

A need exists for a rapid molecular assay to diagnose patients with suspected SARS-CoV-2, to aid in COVID-19 diagnosis, and for future surveillance and epidemiology. The emergence and rapid spread of SARS-CoV-2 to numerous areas throughout the world, has necessitated preparedness and response in public health laboratories, as well as health care and other areas of society in general. The availability of specific and sensitive assays for the detection of the virus are essential for accurate diagnosis of cases, assessment of the extent of the outbreak, monitoring of intervention strategies and surveillance studies. The disclosed oligonucleotides, methods, and kits can be used in an assay to detect the presence or absence of SARS-CoV-2 virus in a biological sample and to aid in diagnosis of a subject as having COVID-19 disease, thereby informing treatment decisions for the subject. The disclosed assays target specific nucleic acid sequences from the genome of the SARS-CoV-2 virus, in particular, regions in the nucleocapsid protein (N protein) gene and spike protein (S protein) gene of SARS-CoV-2. By targeting one or more regions of the SARS-CoV-2 virus RNA, the assays differentiate SARS-CoV-2 from other clinically relevant non-SARS-CoV-2 coronaviruses.

Accordingly, in some aspects, the disclosure relates to oligonucleotides (having a 5′ terminus and a 3′ terminus) that recognize regions in the N protein gene or the S protein gene of SARS-CoV-2. The nucleotide sequence of the oligonucleotide consists of 42 or less nucleotides and has a nucleotide sequence that consists essentially of, or is a variant of, the nucleotide sequence of: SEQ ID NO:1, SEQ ID NO:2, SEQ ID NO:3, SEQ ID NO:5, SEQ ID NO:6, or SEQ ID NO:7. In some aspects, the variant thereof has no more than 5 substitutions, deletions, or additions. In some embodiments, the oligonucleotide is modified with an internal spacer or a detectable label, for example, when the nucleotide sequence of the oligonucleotide comprises SEQ ID NO:3 or SEQ ID NO:7. In some embodiments, the 5′ terminus is labeled with a fluorophore and the 3′ terminus is complexed to a quencher of fluorescence of said fluorophore. In certain embodiments, the nucleotide sequence of the oligonucleotide further comprises a universal tail sequence, for example, a sequence selected from SEQ ID NO:13 and SEQ ID NO:14.

The kits described herein comprises a primer pair, wherein at least one primer of the primer pair consists of less than 42 nucleotides and has a nucleotide sequence that consists essentially of, or is a variant of, the nucleotide sequence of: SEQ ID NO:1, SEQ ID NO:2, SEQ ID NO:5, or SEQ ID NO:6, and wherein the primer pair is capable of detecting SARS-CoV-2, if present, in the sample by amplification; and SARS-CoV-2 detection reagents. In some aspects, the nucleotide sequence of the variant has no more than 5 substitutions, deletions, or additions when compared to the nucleotide sequence of SEQ ID NO:1, SEQ ID NO:2, SEQ ID NO:5, or SEQ ID NO:6. In some embodiments, the at least one of the primers of the primer pair is modified with an internal spacer or a detectable label. In certain embodiments, the kit further comprises a probe modified with an internal spacer or detectable label. The probe hybridizes to an oligonucleotide having a nucleotide sequence that consists essentially of SEQ ID NO:4 or SEQ ID NO:8. In some aspects, the probe is labeled with a fluorophore and a quencher of fluorescence of the fluorophore.

In particular embodiments of the kit, SEQ ID NO:1 and SEQ ID NO:2 make the primer pair. In other embodiments, SEQ ID NO:5 and SEQ ID NO:6 make the primer pair. In still other embodiments, the kit comprises two primer pairs, which are made of SEQ ID NO:1 and SEQ ID NO:2 for one pair and SEQ ID NO:5 and SEQ ID NO:6 for the other pair.

The kit may further comprise running buffer and a test strip. The test strip comprises filter paper and/or chitosan.

In particular embodiments, the kit comprises a first forward primer comprising SEQ ID NO: 1, a first reverse primer comprising SEQ ID NO: 2, a detectably labeled first probe comprising SEQ ID NO: 3, a second forward primer comprising SEQ ID NO: 5, a second reverse primer comprising SEQ ID NO: 6, a detectably labeled second probe comprising SEQ ID NO: 7, and optionally one or more PCR reagents. The first forward primer, the first reverse primer, the detectably labeled first probe, the second forward primer, the second reverse primer, the detectably labeled second probe, and the one or more PCR reagents may be lyophilized. The kit may further comprise an indication of a result that signifies the presence of SARS-CoV-2 and an indication of a result that signifies the absence of SARS-CoV-2. The result may comprise a Ct value or a Cq value.

In certain embodiments, the kit comprises two primer pairs. The sequence of one primer of the first primer pair consists essentially of: SEQ ID NO:1, SEQ ID NO:1 and a universal tail sequence, or SEQ ID NO:9. The sequence of the other primer of the first primer pair consists essentially of: SEQ ID NO:2, SEQ ID NO:2 and a universal tail sequence, or SEQ ID NO:10. The sequence of one primer of the second primer pair consists essentially of: SEQ ID NO:5, SEQ ID NO:5 and a universal tail sequence, or SEQ ID NO:11. The sequence of the other primer of the second primer pair consists essentially of: SEQ ID NO:6, SEQ ID NO:6 and a universal tail sequence, or SEQ ID NO:12.

The methods described herein comprise mixing the biological sample in vitro with a primer pair that is capable of amplifying a SARS-CoV-2 amplicon product, if the SARS-CoV-2 polynucleotide is present in the biological sample, and amplifying the SARS-CoV-2 amplicon product. At least one primer of the primer pair consists of 42 or less nucleotides and has a nucleotide sequence that consists essentially of, or is a variant of, the nucleotide sequence of: SEQ ID NO:1, SEQ ID NO:2, SEQ ID NO:5, or SEQ ID NO:6. In some implementations, the nucleotide sequence of the variant has no more than 5 substitutions, deletions, or additions when compared to the nucleotide sequence of SEQ ID NO:1, SEQ ID NO:2, SEQ ID NO:5, or SEQ ID NO:6. In some implementations, the primer pair comprises a first primer pair that amplifies a N protein gene amplicon product of SARS-CoV-2 and a second primer pair that amplifies a S protein gene amplicon product of SARS-CoV-2. For example, the primer pair consists of: SEQ ID NO:1 and SEQ ID NO:2; or SEQ ID NO:5 and SEQ ID NO:6. In some implementations, the primer pair includes at least two primer pairs comprising SEQ ID NO:1 and SEQ ID NO:2; and SEQ ID NO:5 and SEQ ID NO:6. In some aspects, the amplicon product has a nucleotide sequence that consists essentially of SEQ ID NO:4 or SEQ ID NO:8.

The method further comprises contacting the SARS-CoV-2 amplicon product with a probe having a nucleotide sequence capable of hybridizing to the SARS-CoV-2 amplicon product, the probe being modified with an internal spacer or detectable label, and detecting whether SARS-CoV-2 polynucleotides are present in the biological sample by detecting the detectable label when the probe hybridizes to the SARS-CoV-2 amplicon. In particular implementations, the nucleotide sequence of the probe comprises the sequence of SEQ ID NO:3 or SEQ ID NO:7. In some aspects, the probe is labeled with a fluorophore and a quencher of fluorescence of the fluorophore. The nucleic acid amplification may comprise calculating a Ct value or a Cq value.

In some embodiments, the biological sample comprises a nasopharyngeal swab sample or sputum. In some aspects, the biological sample is from a human.

In particular embodiments of the methods, two primer pairs are mixed with the biological sample. The sequence of one primer of the first primer pair consists essentially of: SEQ ID NO:1, SEQ ID NO:1 and a universal tail sequence, or SEQ ID NO:9. The sequence of the other primer of the first primer pair consists essentially of: SEQ ID NO:2, SEQ ID NO:2 and a universal tail sequence, or SEQ ID NO:10. The sequence of one primer of the second primer pair consists essentially of: SEQ ID NO:5, SEQ ID NO:5 and a universal tail sequence, or SEQ ID NO:11. The sequence of the other primer of the second primer pair consists essentially of: SEQ ID NO:6, SEQ ID NO:6 and a universal tail sequence, or SEQ ID NO:12. Where the sequence of one primer of the first primer pair consists essentially of SEQ ID NO:9 or SEQ ID NO:1 and a universal tail sequence; the sequence of the other primer of the first primer pair consists essentially of SEQ ID NO:10 or SEQ ID NO:2 and a universal tail sequence; the sequence of one primer of the second primer pair consists essentially of SEQ ID NO:11 or SEQ ID NO:5 and a universal tail sequence; and the sequence of the other primer of the second primer pair consists essentially of SEQ ID NO:12 or SEQ ID NO:6 and a universal tail sequence, the method may further comprise analyzing the nucleic acid amplification products by sequencing the nucleic acid amplification products using next-generation sequencing. Accordingly, the method also further comprises adding an index to the nucleic acid amplification products using at least one indexing oligonucleotide. In some aspects, the at least one indexing oligonucleotide comprises a complementary sequence that recognizes the universal tail sequence, SEQ ID NO:13, or SEQ ID NO:14.

In some implementations, the method of detecting SARS-CoV-2 in a subject may include the steps of adding to a mixture containing a sample from the subject, (a) a first forward primer comprising SEQ ID NO: 1, (b) a first reverse primer comprising SEQ ID NO: 2, (c) a second forward primer comprising SEQ ID NO: 5, and (d) a second reverse primer comprising SEQ ID NO: 6, subjecting the mixture to conditions that allow nucleic acid amplification, and detecting the presence or absence of SARS-CoV-2 by analyzing the nucleic acid amplification products. In various embodiments, the method further comprises adding to the mixture a detectably labeled first probe comprising SEQ ID NO: 3 and a detectably labeled second probe comprising SEQ ID NO: 7, and detecting the detectably labeled first probe and the detectably labeled second probe, thereby detecting the presence of SARS-CoV-2 in the subject. In various embodiments, the first forward primer and the second forward primer may further include a first universal tail sequence comprising SEQ ID NO: 13, and wherein the first reverse primer and the second reverse primer include a second universal tail sequence comprising SEQ ID NO: 14. The method may further comprise adding an index to the nucleic acid amplification products using at least one indexing oligonucleotide. The method may further comprise analyzing the nucleic acid amplification products by sequencing the nucleic acid amplification products using next-generation sequencing.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

The present invention relates to methods and kits for assaying for the presence of SARS-CoV-2 in a sample and to oligonucleotides, reagents and kits useful in such assays. The methods, kits, and oligonucleotides are specific for detecting SARS-CoV-2. The disclosed methods and assays detect SARS-CoV-2 RNA, in particular RNA encoding the nucleocapsid protein (N protein) or the spike protein (S protein).

As used herein, the term “sample” (or specimen) may refer to any source in which coronavirus nucleic acids may be detectable. A sample may be derived from anywhere that a virus may be found including soil, air, water, solid surfaces (whether natural or artificial,) culture media, foodstuffs, and any interfaces between or combinations of these elements. Thus, a sample may be an environmental sample or a biological sample, such as a sample obtained from a subject. As used herein, a biological sample includes cells, tissues, and bodily fluids, such as: blood; derivatives and fractions of blood, such as plasma or serum; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; bone marrow aspirates; lymph fluid; ascites; serous fluid; pleural effusion; semen; amniotic fluid; stool; or hair. Samples may be collected by any method now known or yet to be disclosed, including swiping or swabbing an area or orifice, removal of a piece of tissue as in a biopsy, or any method known to collect bodily fluids. In some aspects, a biological sample includes nasal swab, nasopharyngeal swab, bronchial wash, or bronchioalveolar lavage fluid (BALF) from a subject. As used herein, the term “subject” refers includes humans or animals. Emphasis must be placed on the timely collection and appropriate handling of patient samples in order to increase the likelihood of detection of RNA viruses, in this case SARS-CoV-2 detection.

As used herein, the method of or the assay, kit, or oligonucleotides for the detection of SARS-CoV-2 is “specific” for SARS-CoV-2 if the method or the assay using the kit or oligonucleotides can be conducted under conditions that permit the detection of SARS-CoV-2 without exhibiting cross-reactivity to human DNA, or to DNA (or cDNA) of other pathogens, especially other coronavirus pathogens. In particular, an assay for the detection of SARS-CoV-2 is specific for SARS-CoV-2 if it can be conducted under conditions that permit it to detect SARS-CoV-2 without exhibiting cross-reactivity to DNA (or cDNA) of other commonly known human respiratory pathogens or the diverse microbial population in a typical human respiratory tract. More preferably, the assay for the detection of SARS-CoV-2 is said to be specific for SARS-CoV-2 if it can be conducted under conditions that permit it to detect SARS-CoV-2 without exhibiting cross-reactivity to DNA (or cDNA) of SARS-CoV, MERS-CoV, human coronaviruses 229E, OC43, HKU1, or NL63, adenovirus, human metapneumovirus, parainfluenza virus 1-4, Influenza A, Influenza B, enterovirus, respiratory syncytial virus (RSV), rhinovirus, Chlamydophila pneumoniae, Haemophilus influenzae, Legionella pneumophila, Mycobacterium tuberculosis, Streptococcus pneumoniae, Streptococcus pyogenes, Bordetella pertussis, Mycoplasma pneumoniae, Pneumocystis jirovecii, Candida albicans, Pseudomonas aeruginosa, Staphylococcus epidermis, Staphylococcus salivarius, or pooled human nasal fluid.

The methods and assays described herein are for the detection of SARS-CoV-2 in a sample in vitro. The disclosed methods and assays include polymerase chain reaction (PCR) test for the detection of nucleic acid from the SARS-CoV-2 virus. In particular embodiments, the disclosed methods and assays include a real-time reverse transcription PCR (rRT-PCR) test for the qualitative detection of nucleic acid from the SARS-CoV-2 virus. The disclosed SARS-CoV-2 primer and probe sets are designed to detect RNA from the SARS-CoV-2 virus in biological samples from patients, such as patients suspected of having COVID-19.

In some implementations, the biological sample is pre-treated to extract RNA that may be present in the sample. Alternatively, the sample is evaluated without prior RNA extraction. For example, rRT-PCR assays of the present invention may be envisioned as involving multiple reaction steps:

(1) the reverse transcription of SARS-CoV-2 RNA that may be present in the clinical sample that is to be evaluated for SARS-CoV-2 presence;

(2) the PCR-mediated amplification of the SARS-CoV-2 cDNA produced from such reverse transcription;

(3) the hybridization of SARS-CoV-2-specific probes to such amplification products;

(4) the double-strand-dependent 5′→3′ exonuclease cleavage of the hybridized SARS-CoV-2-specific probes; and

(5) the detection of the unquenched probe fluorophores signifying that the evaluated clinical sample contained SARS-CoV-2.

It will be understood that such steps may be conducted separately (for example, in two or more reaction chambers, or with reagents for the different steps being added at differing times, etc.). However, it is preferred that such steps are to be conducted within the same reaction chamber, and that all reagents needed for the rRT-PCR assays of the present invention are to be provided to the reaction chamber at the start of the assay. It will also be understood that although the PCR is the preferred method of amplifying SARS-CoV-2 cDNA produced via reverse transcription, other DNA amplification technologies could alternatively be employed.

Accordingly, in a preferred embodiment, the rRT-PCR assays of the present invention comprise incubating a clinical sample in the presence of a DNA polymerase, a reverse transcriptase, one or more pairs of SARS-CoV-2-specific primers, one or more SARS-CoV-2-specific probes (typically, at least one probe for each region being amplified by an employed pair of primers), deoxynucleotide triphosphates (dNTPs) and buffers. The conditions of the incubation are cycled to permit the reverse transcription of SARS-CoV-2 RNA, the amplification of SARS-CoV-2 cDNA, the hybridization of SARS-CoV-2-specific probes to such cDNA, the cleavage of the hybridized SARS-CoV-2-specific probes and the detection of unquenched probe fluorophores.

The primer pair comprises a forward primer that hybridizes to a polynucleotide portion of a first strand of a DNA molecule and a reverse primer that hybridizes to a polynucleotide portion of a second (and complementary) strand of such DNA molecule. The forward and reverse primers will permit the amplification of a region of the N protein gene or a region of the S protein gene. The amplification of either of such targets alone is sufficient for the specific determination of SARS-CoV-2 presence in clinical samples. It is, however, preferred to assay for SARS-CoV-2 by amplifying both such targets for improved confidence in the assay results.

The presence of such amplified molecules is preferably detected using probes that are capable of hybridizing to an oligonucleotide region present within the oligonucleotide that is amplified by the above-described SARS-CoV-2-specific primers. Such detection can be accomplished using any suitable method, e.g., molecular beacon probes, scorpion primer-probes, TaqMan® probes, etc. All of these methods employ an oligonucleotide that is labeled with a fluorophore and complexed to a quencher of the fluorescence of that fluorophore.

A wide variety of fluorophores and quenchers are known and are commercially available and may be used in accordance with the methods of the present invention. Preferred fluorophores include the fluorophores Biosearch Blue, Alexa488, FAM, Oregon Green, Rhodamine Green-X, NBD-X, TET, Alexa430, BODIPY R6G-X, CAL Fluor Gold 540, JOE, Yakima Yellow, Alexa 532, VIC, HEX, and CAL Fluor Orange 560 (which have an excitation wavelength in the range of about 352-538 nm and an emission wavelength in the range of about 447-559 nm, and whose fluorescence can be quenched with the quencher BHQ1), or the fluorophores RBG, Alexa555, BODIPY 564/570, BODIPY TMR-X, Quasar 570, Cy3, Alexa 546, NED, TAMRA, Rhodamine Red-X, BODIPY 581/591, Redmond Red, CAL Fluor Red 590, Cy3.5, ROX, Alexa 568, CAL Fluor Red 610, BODIPY TR-X, Texas Red, CAL Fluor Red 635, Pulsar 650, Cy5, Quasar 670, CY5.5, Alexa 594, BODIPY 630/650-X, or Quasar 705 (which have an excitation wavelength in the range of about 524-690 nm and an emission wavelength in the range of about 557-705 nm, and whose fluorescence can be quenched with the quencher BHQ2). The preferred SARS-CoV-2-specific TaqMan probes of the present invention are labeled with either the fluorophore 2′,7′-dimethoxy-4′,5′-dichloro-6-carboxyfluorescein (“JOE”) or the fluorophore 5(6)-carboxyfluorescein (“FAM”) on their 5′ termini. JOE is a xanthene fluorophore with an emission in yellow range (absorption wavelength of 520 nm; emission wavelength of 548 nm). FAM is a carboxyfluorescein molecule with an absorption wavelength of 495 nm and an emission wavelength of 517 nm; it is typically provided as a mixture of two isomers (5-FAM and 6-FAM). Quasar 670 is similar to cyanine dyes, and has an absorption wavelength of 647 nm and an emission wavelength of 670 nm.

The black hole quencher 1 (“BHQ1”) is a preferred quencher for FAM and JOE fluorophores. BHQ1 quenches fluorescent signals of 480-580 nm and has an absorption maximum at 534 nm.

The black hole quencher 2 (“BHQ2”) is a preferred quencher for Quasar 670. BHQ2 quenches fluorescent signals of 560-670 nm and has an absorption maximum at 579 nm.

JOE, FAM, Quasar 670, BHQ1 and BHQ2 are widely available commercially and are coupled to oligonucleotides using methods that are well known. Oligonucleotide probes of any desired sequence labeled may be obtained commercially already labeled with a desired fluorophore and complexed with a desired quencher.

As discussed above, the proximity of the quencher of a TaqMan® probe to the fluorophore of the probe results in a quenching of the fluorescent signal. Incubation of the probe in the presence of a double-strand-dependent 5′→3′ exonuclease (such as the 5″→3″ exonuclease activity of Taq polymerase) cleaves the probe when it has hybridized to a complementary target sequence, thus separating the fluorophore from the quencher and permitting the production of a detectable fluorescent signal.

Molecular beacon probes can alternatively be employed to detect amplified SARS-CoV-2 oligonucleotides in accordance with the present invention. Molecular beacon probes are also labeled with a fluorophore and complexed to a quencher. However, in such probes, the quenching of the fluorescence of the fluorophore only occurs when the quencher is directly adjacent to the fluorophore. Molecular beacon probes are thus designed to adopt a hairpin structure while free in solution (thus bringing the fluorescent dye and quencher into close proximity with one another). When a molecular beacon probe hybridizes to a target, the fluorophore is separated from the quencher, and the fluorescence of the fluorophore becomes detectable. Unlike TaqMan probes, molecular beacon probes are designed to remain intact during the amplification reaction, and must rebind to target in every cycle for signal measurement.

Scorpion primer-probes can alternatively be employed to detect amplified SARS-CoV-2 oligonucleotides in accordance with the present invention. Scorpion primer-probes are also designed to adopt a hairpin structure while free in solution and are also labeled with a fluorophore at their 5′ terminus and complexed to a quencher at their 3′ terminus. Scorpion primer-probes differ from molecular beacon probes in that their 3′-end is attached to their 5′-end by a hexathylene glycol (HEG) blocker. Such attachment prevents the polymerase-mediated extension of the 3′ terminus of the scorpion primer-probe. However, after the scorpion primer-probe has bound to its target DNA, the polymerase copies the sequence of nucleotides from its 3′-end. In the next denaturation step, the specific sequence of the scorpion primer-probe binds to the complementary region within the same strand of newly amplified DNA. This hybridization opens the hairpin structure and, as a result, separates the molecules fluorophore from its quencher and permits fluorescence to be detected.

In a preferred embodiment, the probes of the present invention are TaqMa® probes. As described above, such probes are labeled on their 5′ termini with a fluorophore and are complexed on their 3′ termini with a quencher of the fluorescence of that fluorophore. In order to simultaneously detect the amplification of two polynucleotide portions of SARS-CoV-2, two TaqMan probes are employed that have different fluorophores (with differing and distinguishable emission wavelengths); the employed quenchers may be the same or different. In one embodiment of the invention, the 5′ terminus of the first probe is labeled with the fluorophore JOE, and the 3′ terminus of such probe is complexed to the quencher BHQ1 and the 5′ terminus of the second probe is labeled with the fluorophore FAM, and the 3′ terminus of such probe is complexed to the quencher BHQ1. In an alternative embodiment, the 5′ terminus of the first probe is labeled with the fluorophore FAM, and the 5′ terminus of the second probe is labeled with the fluorophore JOE. The use of such two fluorophores permits both probes to be used in the same assay.

The rRT-PCR assay described herein comprises one or more pairs of primers that amplify regions of the N protein and/or the S protein of SARS-CoV-2. In one embodiment, the assay comprises a first primer pair and probe targeting the nucleocapsid protein gene (N protein gene) of SARS-CoV-2 and a second primer pair and probe targeting the spike protein gene (S protein gene) of SARS-CoV-2. The methods of detecting SARS-CoV-2 in a sample in vitro comprise mixing the biological sample in vitro with a primer pair that is capable of amplifying a SARS-CoV-2 amplicon product, if the SARS-CoV-2 polynucleotide is present in the biological sample, and amplifying the SARS-CoV-2 amplicon product. At least one primer of the primer pair consists of 42 or less nucleotides and has a nucleotide sequence that consists essentially of, or is a variant of, the nucleotide sequence of: SEQ ID NO:1, SEQ ID NO:2, SEQ ID NO:5, or SEQ ID NO:6. In some implementations, the nucleotide sequence of the variant has no more than 5 substitutions, deletions, or additions when compared to the nucleotide sequence of SEQ ID NO:1, SEQ ID NO:2, SEQ ID NO:5, or SEQ ID NO:6. In some implementations, the primer pair comprises a first primer pair that amplifies a N protein gene amplicon product of SARS-CoV-2 and a second primer pair that amplifies a S protein gene amplicon product of SARS-CoV-2. For example, the primer pair consists of: SEQ ID NO:1 and SEQ ID NO:2; or SEQ ID NO:5 and SEQ ID NO:6. In some implementations, the primer pair includes at least two primer pairs comprising SEQ ID NO:1 and SEQ ID NO:2; and SEQ ID NO:5 and SEQ ID NO:6. In some aspects, the amplicon product has a nucleotide sequence that consists essentially of SEQ ID NO:4 or SEQ ID NO:8.

The method further comprises contacting the SARS-CoV-2 amplicon product with a probe having a nucleotide sequence capable of hybridizing to the SARS-CoV-2 amplicon product, the probe being modified with an internal spacer or detectable label, and detecting whether SARS-CoV-2 polynucleotides are present in the biological sample by detecting the detectable label when the probe hybridizes to the SARS-CoV-2 amplicon. In particular implementations, the nucleotide sequence of the probe comprises the sequence of SEQ ID NO:3 or SEQ ID NO:7. In some aspects, the probe is labeled with a fluorophore and a quencher of fluorescence of the fluorophore. The nucleic acid amplification may comprise calculating a Ct value or a Cq value.

In some embodiments, the biological sample comprises a nasopharyngeal swab sample or sputum. In some aspects, the biological sample is from a human.

In particular embodiments of the methods, two primer pairs are mixed with the biological sample. The sequence of one primer of the first primer pair consists essentially of: SEQ ID NO:1, SEQ ID NO:1 and a universal tail sequence, or SEQ ID NO:9. The sequence of the other primer of the first primer pair consists essentially of: SEQ ID NO:2, SEQ ID NO:2 and a universal tail sequence, or SEQ ID NO:10. The sequence of one primer of the second primer pair consists essentially of: SEQ ID NO:5, SEQ ID NO:5 and a universal tail sequence, or SEQ ID NO:11. The sequence of the other primer of the second primer pair consists essentially of: SEQ ID NO:6, SEQ ID NO:6 and a universal tail sequence, or SEQ ID NO:12. Where the sequence of one primer of the first primer pair consists essentially of SEQ ID NO:9 or SEQ ID NO:1 and a universal tail sequence; the sequence of the other primer of the first primer pair consists essentially of SEQ ID NO:10 or SEQ ID NO:2 and a universal tail sequence; the sequence of one primer of the second primer pair consists essentially of SEQ ID NO:11 or SEQ ID NO:5 and a universal tail sequence; and the sequence of the other primer of the second primer pair consists essentially of SEQ ID NO:12 or SEQ ID NO:6 and a universal tail sequence, the method may further comprise analyzing the nucleic acid amplification products by sequencing the nucleic acid amplification products using next-generation sequencing. Accordingly, the method also further comprises adding an index to the nucleic acid amplification products using at least one indexing oligonucleotide. In some aspects, the at least one indexing oligonucleotide comprises a complementary sequence that recognizes the universal tail sequence, SEQ ID NO:13, or SEQ ID NO:14.

In some implementations, the method of detecting SARS-CoV-2 in a subject may include the steps of adding to a mixture containing a sample from the subject, (a) a first forward primer comprising SEQ ID NO: 1, (b) a first reverse primer comprising SEQ ID NO: 2, (c) a second forward primer comprising SEQ ID NO: 5, and (d) a second reverse primer comprising SEQ ID NO: 6, subjecting the mixture to conditions that allow nucleic acid amplification, and detecting the presence or absence of SARS-CoV-2 by analyzing the nucleic acid amplification products. In various embodiments, the method further comprises adding to the mixture a detectably labeled first probe comprising SEQ ID NO: 3 and a detectably labeled second probe comprising SEQ ID NO: 7, and detecting the detectably labeled first probe and the detectably labeled second probe, thereby detecting the presence of SARS-CoV-2 in the subject. In various embodiments, the first forward primer and the second forward primer may further include a first universal tail sequence comprising SEQ ID NO: 13, and wherein the first reverse primer and the second reverse primer include a second universal tail sequence comprising SEQ ID NO: 14. The method may further comprise adding an index to the nucleic acid amplification products using at least one indexing oligonucleotide. The method may further comprise analyzing the nucleic acid amplification products by sequencing the nucleic acid amplification products using next-generation sequencing.

TABLE 1 shows the primers and probes for a real-time PCR assay targeting the N protein gene and the spike protein gene of SARS-CoV-2. The “TG-N2” assay targets a 78 bp region of the N protein gene. The “CoV-TGS01” assay targets a 77 bp region of the spike protein gene of SARS-CoV-2.

While one assay (one primer pair and probes described herein) detects the presence or absence of SARS-CoV-2 virus, the use of two assays (two primer pairs and their respective probe) targeting different genes detects the presence or absence of SARS-CoV-2 virus with greater reliability than a single assay used alone. The TG-N2 assay and the TG-S4 assay (also referred to herein as the “SARS-CoV-2 rRT-PCR Assay”) detect the presence or absence of SARS-CoV-2 virus with greater reliability than either the TG-N2 assay or the TG-S4 assay used alone. In further embodiments, two or more SARS-CoV-2 assays (primer pairs and probes), where at least one first assay targets the N protein gene of SARS-CoV-2 and where at least one second assay targets the spike protein gene of SARS-CoV-2, may detect the presence or absence of SARS-CoV-2 virus with greater reliability than an assay or combination of assays that target only one of the N protein gene or the S protein gene of SARS-CoV-2.

In various embodiments, the SARS-CoV-2 rRT-PCR assay comprises two forward primers (SEQ ID NO: 1 and SEQ ID NO: 5), two reverse primers (SEQ ID NO: 2 and SEQ ID NO: 6), and two probes (SEQ ID NO: 3 and SEQ ID NO: 7). The TG-N2 assay comprises a forward primer (SEQ ID NO: 1), a reverse primer (SEQ ID NO: 2) and a probe (SEQ ID NO: 3). The CoV-TS01 assay comprises a forward primer (SEQ ID NO: 5), a reverse primer (SEQ ID NO: 6) and a probe (SEQ ID NO: 7).

TABLE 1 also shows sequences of the amplification products of the TG-N2 assay and the CoV-TS01 assay. The amplicon produced using the TG-N2 assay (SEQ ID NO: 1 and SEQ ID NO: 2) has a sequence comprising SEQ ID NO: 4. The amplicon produced using the CoV-TS01 assay (SEQ ID NO: 5 and SEQ ID NO: 6) has a sequence comprising SEQ ID NO: 8.

TABLE 1

| | Assay | | | SEQ |
|---|---|---|---|---|
| | Component | Name | Sequence | ID NO: |
| N protein | forward | TG-N2_F | TTCAGCGTTCTTCGGAATGTC | 1 |
| gene: | primer | | | |
| TG-N2 | reverse | TG-N2_R | TGGCACCTGTGTAGGTCAAC | 2 |
| assay | primer | | | |
| | probe | TG-N2_ | CGCATTGGCATGGAAGTCACA | 3 |
| | | FAMBHQ | CC | |
| | amplicon | Amplicon | TTCAGCGTTCTTCGGAATGTCG | 4 |
| | sequence | Sequence | CGCATTGGCATGGAAGTCACA | |
| | | | CCTTCGGGAACGTGGTTGACC | |
| | | | TACACAGGTGCCA | |
| S protein | forward | CoV-TGS01_F | GCACCTCATGGTGTAGTCTTCT | 5 |
| gene: | primer | | TG | |
| CoV- | reverse | CoV-TGS01_R | TGGCAGGAGCAGTTGTGAA | 6 |
| TGS01 | primer | | | |
| assay | probe | CoV-TGS01_ | CATGTGACTTATGTCCCTGCAC | 7 |
| | | FAMBHQ | AAGAA | |
| | amplicon | Amplicon | GCACCTCATG GTGTAGTCTT | 8 |
| | sequence | Sequence | CTTGCATGTG ACTTATGTCC | |
| | | | CTGCACAAGA AAAGAACTTC | |
| | | | ACAACTGCTC CTGCCA | |

The preferred primers and probes described are designed for the specific detection of SARS-CoV-2. Each target on its own has been shown to provide sensitive and specific detection of SARS-CoV-2 with no detection of, or cross-reactivity to, other coronaviruses. Thus, the invention encompasses oligonucleotides of less than 42 nucleotides in length with nucleotide sequences of these oligonucleotides consisting of, consisting essentially of, or are “variants” of such preferred primers and probes. Thus, these oligonucleotides have a 5′ terminus and a 3′ terminus, recognize regions in the N protein gene or the S protein gene of SARS-CoV-2, and have a nucleotide sequence that consists essentially of, or is a variant of, the nucleotide sequence of: SEQ ID NO:1, SEQ ID NO:2, SEQ ID NO:3, SEQ ID NO:5, SEQ ID NO:6, or SEQ ID NO:7. As used herein, an oligonucleotide is a “variant” of another oligonucleotide if it retains the function of such oligonucleotide (e.g., acting as a specific primer or probe), but:

(1) lacks 1, 2, 3, 4, 5, 6, 7, 8, 9, or 10 nucleotides of the nucleotides of such primer or probe, or

(2) lacks 1, 2, 3, 4, 5, 6, 7, 8, 9, or 10 of the 10 3′ terminal nucleotides of such primer or probe, or

(3) lacks 1, 2, 3, 4, 5, 6, 7, 8, 9, or 10 of the 10 5′ terminal nucleotides of such primer or probe, or

(4) has a sequence that differs from that of such primer or probe in having 1, 2, 3, 4, 5, 6, 7, 8, 9, 10 or more than 10 additional nucleotides, or

(5) has a sequence that differs from that of such primer or probe in having 1, 2, 3, 4, 5, 6, 7, 8, 9, 10 or more than 10 substitution nucleotides in lieu of the nucleotides present in such primer or probe, or

(6) possesses a combination of such (1)-(5).

In some aspects, the variant thereof has no more than 5 substitutions, deletions, or additions. In some embodiments, the oligonucleotide is modified with an internal spacer or a detectable label, for example, when the nucleotide sequence of the oligonucleotide comprises SEQ ID NO:3 or SEQ ID NO:7. In some embodiments, the 5′ terminus is labeled with a fluorophore and the 3′ terminus is complexed to a quencher of fluorescence of said fluorophore. In certain embodiments, the nucleotide sequence of the oligonucleotide further comprises a universal tail sequence, for example, a sequence selected from SEQ ID NO:13 and SEQ ID NO:14.

The disclose also provides kits for detecting SARS-CoV-2 in biological samples. A “kit,” as used herein, refers to a combination of at least some items for performing a PCR assay for coronavirus detection, and more particularly coronavirus strain differentiation, and more particularly SARS-CoV-2 detection. Embodiments of kits may comprise one or more of the following reagents: at least one set of primers specific for SARS-CoV-2 detection, at least one probe specific for SARS-CoV-2 detection, internal positive control DNA to monitor presence of PCR inhibitors from various food and environmental sources, a baseline control, reagents for sample collection, reagents for isolating nucleic acid such as magnetic beads, spin columns, lysis buffers, proteases, reagents for PCR amplification such as a DNA polymerase or an enzymatically active mutant or variant thereof, reverse transcriptase, a DNA polymerase buffer, buffer containing dNTPs, deoxyribonucleotides dATP, dCTP, dGTP, or dTTP. In some embodiments, a probe is a TaqMan® probe. In certain kit embodiments, amplification primers are attached to a solid support such as a microarray. In some embodiments, a kit may include an internal control (for example, RNase P assay).

One or more kit components may be packaged in one or more container means. Kit container means may generally include at least one vial, test tube, flask, bottle, syringe or other packaging means, into which a component can be placed, and in some embodiments, suitably aliquoted. Where more than one component is included in a kit (they can be packaged together), the kit also will generally contain at least one second, third or other additional container into which the additional components can be separately placed.

However, various combinations of components can be packaged in a container means. Kits of the present teachings also will typically include reagent containers in close confinement for commercial sale. Such containers can include injection or blow-molded plastic containers into which the desired container means are retained. When the components of kits are provided in one and/or more liquid solutions, the liquid solution comprises an aqueous solution that can be a sterile aqueous solution.

In certain embodiments, at least one kit component is lyophilized and provided as dried powder(s). For example, primers and TaqMan® probes may be lyophilized. When reagents and/or components are provided as a dry powder, the powder can be reconstituted by the addition of a suitable solvent. In certain embodiments, a solvent is provided in another container means. Kits can also comprise an additional container means for containing a sterile, pharmaceutically acceptable buffer and/or other diluent.

A kit can also include instructions for employing the kit components as well as the use of any other reagent not included in the kit. Instructions can include variations that can be implemented. A kit may also contain an indication that links the output of the kit to a particular result. For example, an indication may be one or more sequences or that signify the identification of a particular fungal phylum, class, order, family, genus species, subspecies, strain or any other delineation of a group of fungi. An indication may include a Ct value, wherein exceeding the Ct value indicates the presence or absence of an organism of interest. A kit may contain a positive control. A kit may contain a standard curve configured to quantify the amount of fungus present in a sample. An indication includes any guide that links the output of the kit to a particular result. The indication may be a level of fluorescence or radioactive decay, a value derived from a standard curve, or from a control, or any combination of these and other outputs. The indication may be printed on a writing that may be included in the kit or it may be posted on the Internet or embedded in a software package.

In particular embodiments, the kit comprises a primer pair, wherein at least one primer of the primer pair consists of less than 42 nucleotides and has a nucleotide sequence that consists essentially of, or is a variant of, the nucleotide sequence of: SEQ ID NO:1, SEQ ID NO:2, SEQ ID NO:5, or SEQ ID NO:6, and wherein the primer pair is capable of detecting SARS-CoV-2, if present, in the sample by amplification; and SARS-CoV-2 detection reagents. In some aspects, the nucleotide sequence of the variant has no more than 5 substitutions, deletions, or additions when compared to the nucleotide sequence of SEQ ID NO:1, SEQ ID NO:2, SEQ ID NO:5, or SEQ ID NO:6. In some embodiments, the at least one of the primers of the primer pair is modified with an internal spacer or a detectable label. In certain embodiments, the kit further comprises a probe modified with an internal spacer or detectable label. The probe hybridizes to an oligonucleotide having a nucleotide sequence that consists essentially of SEQ ID NO:4 or SEQ ID NO:8. In some aspects, the probe is labeled with a fluorophore and a quencher of fluorescence of the fluorophore.

In particular embodiments of the kit, SEQ ID NO:1 and SEQ ID NO:2 make the primer pair. In other embodiments, SEQ ID NO:5 and SEQ ID NO:6 make the primer pair. In still other embodiments, the kit comprises two primer pairs, which are made of SEQ ID NO:1 and SEQ ID NO:2 for one pair and SEQ ID NO:5 and SEQ ID NO:6 for the other pair.

The kit may further comprise running buffer and a test strip. The test strip comprises filter paper and/or chitosan.

In particular embodiments, the kit comprises a first forward primer comprising SEQ ID NO:1, a first reverse primer comprising SEQ ID NO:2, a detectably labeled first probe comprising SEQ ID NO:3, a second forward primer comprising SEQ ID NO:5, a second reverse primer comprising SEQ ID NO:6, a detectably labeled second probe comprising SEQ ID NO:7, and optionally one or more PCR reagents. The first forward primer, the first reverse primer, the detectably labeled first probe, the second forward primer, the second reverse primer, the detectably labeled second probe, and the one or more PCR reagents may be lyophilized. The kit may further comprise an indication of a result that signifies the presence of SARS-CoV-2 and an indication of a result that signifies the absence of SARS-CoV-2. The result may comprise a Ct value or a Cq value.

In certain embodiments, the kit comprises two primer pairs. The sequence of one primer of the first primer pair consists essentially of: SEQ ID NO:1, SEQ ID NO:1 and a universal tail sequence, or SEQ ID NO:9. The sequence of the other primer of the first primer pair consists essentially of: SEQ ID NO:2, SEQ ID NO:2 and a universal tail sequence, or SEQ ID NO:10. The sequence of one primer of the second primer pair consists essentially of: SEQ ID NO:5, SEQ ID NO:5 and a universal tail sequence, or SEQ ID NO:11. The sequence of the other primer of the second primer pair consists essentially of: SEQ ID NO:6, SEQ ID NO:6 and a universal tail sequence, or SEQ ID NO:12.

### EXAMPLES

The present invention is further illustrated by the following examples that should not be construed as limiting. The contents of all references, patents, and published patent applications cited throughout this application are incorporated herein by reference in their entirety for all purposes.

1. rRT-PCR with RNA Isolated Using Zymo Kit

The exemplary assay is a rRT-PCR test for the qualitative detection of nucleic acid from the SARS-CoV-2 virus. The disclosed SARS-CoV-2 primer and probe sets are designed to detect RNA from the SARS-CoV-2 virus in respiratory samples from patients, such as patients suspected of having COVID-19. Results show that the disclosed assays detect SARS-CoV-2 RNA.

The SARS-CoV-2 rRT-PCR Assay comprises one or more sets of primers. In one embodiment, the assay comprises a first primer pair and probe targeting the nucleocapsid protein gene (N protein gene) of SARS-CoV-2 and a second primer pair and probe targeting the spike protein gene (S protein gene) of SARS-CoV-2.

a. Test Steps

Clinical samples are processed using the following methods.

Nucleic acids are isolated and purified from nasopharyngeal samples using the Quick DNA/RNAViral Kit (Zymo Research). Sample input volume was 100 and elution volume was 30 μL. RNA isolation is manual and adaptable to several automated liquid handling instruments.

The purified nucleic acid was reverse transcribed into cDNA and subsequently amplified using qScript One-Step RT-qPCR Kit (QuantaBio), in a 20 μL reaction containing 2 of purified RNA from a sample, on the CFX96 Real-Time PCR Detection System (Bio-Rad). In the process, the probe anneals to a specific target sequence located between the forward and reverse primers. During the extension phase of the PCR cycle, the 5′ nuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity is monitored at each PCR cycle by the CFX96 Real-Time PCR Detection System (Bio-Rad).

b. Control Materials

An extraction control comprised of the material from an unused nasopharyngeal sample collection device was used to identify any background or spurious signal that may have come from RNA extraction kit reagents or RNA extraction sample setup and interfered with accurate test result interpretation. The extraction control is used beside each set of samples for each RNA extraction procedure.

Negative Extraction Control: A negative extraction control comprised of the material from a known negative sample, such as Universal Transport Medium, is used to determine that the RNA extraction and rRT-PCR assay are working as expected.

Negative PCR Control: A “no template” or “template-free” (negative) PCR control, or “NTC”, is used to detect any background or spurious real-time PCR fluorescence that may interfere with accurate interpretation of results from samples and multiple no template controls are used for each primer set for each rRT-PCR run.

Positive PCR Control: A positive template PCR control was used to confirm that the rRT-PCR assay and thermal cycling are performing as expected. A positive template PCR control is used for each primer set for each rRT-PCR run. The positive control is precisely quantified SARS-CoV-2 genomic RNA (supplied by BEI Resources), is run at two to three times the LoD of the assay, and is placed next to a no template control to identify if cross-contamination may have occurred during reaction setup.

Positive Extraction Control: An internal control, comprised of the CDC's RNase P detection assay contained in their validated SARS-CoV-2 assay set, which detects human RNA, was used to determine that each sample is of sufficient quality to be included in the SARS-CoV-2 rRT-PCR Assay and is used for each sample.

c. Control Results Interpretation and Quality Control Criteria

In embodiments where two or more assays are used in the detection of SARS-CoV-2, the following criteria are used to determine if the results are positive, negative, or undetermined.

TABLE 2 shows the expected performance of the Negative PCR Control, the Positive PCR Control, the Negative Extraction Control and the Positive Extraction Control.

TABLE 2
Control Results Criteria

| CONTROL TYPE | USED TO MONITOR | TG-N2 | COV-TGS01 | RNASEP |
|---|---|---|---|---|
| Negative PCR | Assay or rRT-PCR reagent | No Ct | No Ct | No Ct |
| | contamination | | | |
| Positive PCR | Proper assay setup, | Ct < 40.0 | Ct < 40.0 | N/A |
| | reagent/assay integrity, rRT- | | | |
| | PCR success | | | |
| Negative extraction | RNA extraction kit reagent | No Ct | No Ct | Ct < 35.0 |
| control | contamination, RNA extraction | | | |
| | procedures | | | |
| Positive extraction | RNA extraction success, | No Ct | No Ct | Ct < 35.0 |
| control | sample quality, rRT-PCR | | | |
| | success | | | |

To validate and allow for interpretation of the results of any other assay, all no template PCR controls for an assay in a given rRT-PCR run need to be negative, i.e., yield no fluorescence signal that crosses the established assay threshold value. If any no template controls yield fluorescence signal that crosses the threshold, the rRT-PCR run for that assay is invalid and that test for all samples are repeated.

The positive PCR control must test positive for an assay (i.e. yield a Ct value below the established cutoff for that assay; in other words, emit fluorescent signal that crosses the established threshold for that assay before the thermal cycle number cutoff or Ct cutoff designated for each assay) in a given rRT-PCR run to validate results of any other assay. If the positive control does not test positive, that rRT-PCR run for that assay is invalid and that test for all samples are repeated.

The negative extraction control must test negative, i.e., yield no fluorescence signal that crosses the established assay threshold value for any assay, to validate the results of any samples processed (RNA extracted) in the set with that extraction control. If any negative extraction controls yield fluorescence signal for any assay, all rRT-PCR assay results for that sample set are invalid and the RNA extraction and testing for those samples are repeated.

The positive extraction control must test negative for both SARS-CoV-2 rRT-PCR Assay primer and probe sets, and positive for the RNase P assay. If the positive extraction control tests positive on either of the SARS-CoV-2 primer and probe sets, or negative on the RNase P assay, all rRT-PCR assay results for that sample set are invalid and the RNA extraction and testing for those samples are repeated.

The internal control assay targeting the human RNase P RNA must yield a positive result (for example, a Ct value <35, as is outlined in the CDC's test kit protocol) to validate all results for that sample, with one exception. If the sample tests positive for both SARS-CoV-2 rRT-PCR Assay primer and probe sets (TG-N2 and CoV-TGS01), the sample is considered positive. If any sample tests negative for RNase P and either or both of the SARS-CoV-2 rRT-PCR Assay primer and probe sets, that sample is reprocessed from RNA extraction through rRT-PCR testing. If a sample tests negative for RNase P on subsequent testing, it is reported as an invalid sample and no SARS-CoV-2 positive or negative diagnosis is given.

After all positive and negative PCR controls and internal controls have been analyzed and determined to be valid and acceptable, the clinical sample results are interpreted and analyzed d. Results

Results of a real-time RT-PCR screening run on the QuantStudio RT-PCR instrument are shown in TABLE 3 for the TG-N2 assay and in TABLE 4 for the CoV-TGS01 assay. TABLE 3 and TABLE 4 show mean Ct values for each assay.

TABLE 3
Results for TG-N2 rRT-PCR Assay using QuantStudio

| | Concentration | Ct | Standard | Coefficient | Number of replicates positive/ |
|---|---|---|---|---|---|
| | of viral RNA | mean | deviation | of variation | Number of replicates tested |
| 1 | 100 | 28.8 | 0.2071 | 0.7% | 3/3 |
| 2 | 50 | 29.67 | 0.1564 | 0.5% | 3/3 |
| 3 | 25 | 30.86 | 0.2442 | 0.8% | 3/3 |
| 4 | 12.5 | 31.61 | 0.4482 | 1.4% | 3/3 |
| 5 | 6.25 | 32.39 | 0.1386 | 0.4% | 3/3 |
| 6 | 3.125 | 33.66 | 0.6276 | 1.9% | 3/3 |
| 7 | 1.56 | 34.85 | 0.7719 | 2.2% | 3/3 |

TABLE 4
Results for CoV-TGS01 rRT-PCR Assay using QuantStudio

| | Concentration | Ct | Standard | Coefficient of | Number of replicates positive/ |
|---|---|---|---|---|---|
| | of viral RNA | mean | deviation | variation | Number of replicates tested |
| 1 | 100 | 30.25 | 0.1743 | 0.6% | 3/3 |
| 2 | 50 | 31.28 | 0.169 | 0.5% | 3/3 |
| 3 | 25 | 32.11 | 0.1721 | 0.5% | 3/3 |
| 4 | 12.5 | 33.25 | 0.3122 | 0.9% | 3/3 |
| 5 | 6.25 | 34.86 | 0.5734 | 1.6% | 3/3 |
| 6 | 3.125 | 37 | 0.7051 | 1.9% | 3/3 |
| 7 | 1.56 | 36.34 | 0.8075 | 2.2% | 3/3 |

i. Positive Result Example 1:

A sample that yields a Ct value (or Cq value) less than a threshold on both the TG-N2 and the CoV-TGS01 primer and probe sets included in the SARS-CoV-2 rRT-PCR Assay is considered positive for the presence of SARS-CoV-2 virus. In various embodiments, the threshold is 35 and a Ct value of less than (<) 35 indicates a positive result, or the threshold is 38 and a Ct value of less than (<) 38 indicates a positive result, or threshold is 40 and a Ct value of less than (<) 40 indicates a positive result, or threshold is 50 and a Ct value of less than (<) 50 indicates a positive result.

For example, if the result for a sample using the TG-N2 assay is a Ct value (or Cq value) that is less than the threshold, and the result for that sample using the CoV-TGS01 primer is a Ct value (or Cq value) that is also less than the threshold, then the sample is positive or SARS-CoV-2.

ii. Positive Result Example 2

A sample that yields a Ct value of less than a first threshold on at least one of the primer and probe sets and that yields a Ct value between a lower threshold and an upper threshold on other primer and probe sets is considered positive for the presence of SARS-CoV-2. In various embodiments, the first threshold is 35 and a Ct value of less than (<) 35 indicates a positive result, or the first threshold is 38 and a Ct value of less than (<) 38 indicates a positive result, or first threshold is 40 and a Ct value of less than (<) 40 indicates a positive result, or first threshold is 50 and a Ct value of less than (<) 50 indicates a positive result. In various embodiments, the lower threshold is 35 and the upper threshold is 38 and a Ct value between 35 and 38 indicates a positive result, or the lower threshold is 35 and the upper threshold is 40 and a Ct value between 35 and 40 indicates a positive result, or the lower threshold is 35 and the upper threshold is 50 and a Ct value between 35 and 50 indicates a positive result.

For example, if the result for a sample using the TG-N2 assay is a Ct value (or Cq value) that is less than the first threshold, and the result for that sample using the CoV-TGS01 primer is a Ct value (or Cq value) that is between the lower threshold and the upper threshold, then the sample is positive or SARS-CoV-2.

iii. Undetermined Result Example 1

A sample that yields a Ct value between a lower and upper threshold on any of the primer and probe sets (and not below the lower threshold on any primer and probe sets) is considered undetermined for the presence of SARS-CoV-2 and is repeated.

In various embodiments, the lower threshold is 35 and the upper threshold is 38 and a Ct value between 35 and 38 indicates an undetermined result, if the other assays do not have a Ct value below the lower threshold (<35).

iv. Undetermined Result Example 2

A sample that yields a Ct value >38 and <40 on multiple primer and probe sets is considered undetermined and is repeated.

If upon subsequent testing a sample yields another undetermined result, it will be reported as undetermined and no positive or negative SARS-CoV-2 diagnosis can be given.

v. Negative Result Example

A sample that yields a Ct value >38 and <40 on only one primer and probe set and does not yield a Ct value for any other primer and probe set, or a sample that does not yield a Ct value of any primer and probe set, is considered negative for the presence of SARS-CoV-2.

The disclosed assay (SARS-CoV-2 rRT-PCR Assay) was also validated to be used with the CFX96 Real-Time PCR Detection System (Bio-Rad) and the CFX Maestro Software (Bio-Rad). Results of a real-time PCR screening run on the BioRad CFX instrument using two different primer concentrations are shown in TABLE 5 for the TG-N2 assay and for the CoV-TGS01 assay.

TABLE 5
Results for TG-N2 and CoV-TGS01 Assays using CFX instrument

| | Primer | | | | | | | |
|---|---|---|---|---|---|---|---|---|
| | concen- | Viral | | | | | | |
| | tration | genome | TG-N2 | | | CoV-TGS01 | | |
| Sample | (nM) | copies | Cq | Mean | SD | Cq | Mean | SD |
| NTC1 | 600 | 0 | NaN | 0.0 | 0.0 | NaN | 0.0 | 0.0 |
| NTC2 | 600 | 0 | NaN | 0.0 | 0.0 | NaN | 0.0 | 0.0 |
| NTC3 | 600 | 0 | NaN | 0.0 | 0.0 | NaN | 0.0 | 0.0 |
| NTC4 | 450 | 0 | NaN | 0.0 | 0.0 | NaN | 0.0 | 0.0 |
| NTC5 | 450 | 0 | NaN | 0.0 | 0.0 | NaN | 0.0 | 0.0 |
| NTC6 | 450 | 0 | NaN | 0.0 | 0.0 | NaN | 0.0 | 0.0 |
| 240-600-1 | 600 | 240 | 30.0 | 30.1 | 0.1 | 30.0 | 30.1 | 0.1 |
| 240-600-2 | 600 | 240 | 30.0 | 30.1 | 0.1 | 30.0 | 30.1 | 0.1 |
| 240-600-3 | 600 | 240 | 30.2 | 30.1 | 0.1 | 30.2 | 30.1 | 0.1 |
| 240-450-4 | 450 | 240 | 26.8 | 26.9 | 0.2 | 26.8 | 26.9 | 0.2 |
| 240-450-5 | 450 | 240 | 26.8 | 26.9 | 0.2 | 26.8 | 26.9 | 0.2 |
| 240-450-6 | 450 | 240 | 27.2 | 26.9 | 0.2 | 27.2 | 26.9 | 0.2 |
| 24-600-1 | 600 | 24 | 33.8 | 33.8 | 0.2 | 33.8 | 33.8 | 0.2 |
| 24-600-2 | 600 | 24 | 34.0 | 33.8 | 0.2 | 34.0 | 33.8 | 0.2 |
| 24-600-3 | 600 | 24 | 33.5 | 33.8 | 0.2 | 33.5 | 33.8 | 0.2 |
| 24-450-4 | 450 | 24 | 30.9 | 30.8 | 0.3 | 30.9 | 30.8 | 0.3 |
| 24-450-5 | 450 | 24 | 30.9 | 30.8 | 0.3 | 30.9 | 30.8 | 0.3 |
| 24-450-6 | 450 | 24 | 30.5 | 30.8 | 0.3 | 30.5 | 30.8 | 0.3 |
| 2.4-600-1 | 600 | 2.4 | 39.1 | 38.2 | 1.1 | 39.1 | 38.2 | 1.1 |
| 2.4-600-2 | 600 | 2.4 | 38.4 | 38.2 | 1.1 | 38.4 | 38.2 | 1.1 |
| 2.4-600-3 | 600 | 2.4 | 37.0 | 38.2 | 1.1 | 37.0 | 38.2 | 1.1 |
| 2.4-450-4 | 450 | 2.4 | 34.6 | 34.4 | 0.5 | 34.6 | 34.4 | 0.5 |
| 2.4-450-5 | 450 | 2.4 | 33.9 | 34.4 | 0.5 | 33.9 | 34.4 | 0.5 |
| 2.4-450-6 | 450 | 2.4 | 34.8 | 34.4 | 0.5 | 34.8 | 34.4 | 0.5 |

NTC = no template control;
NaN = no detectable result;
SD = Standard deviation

The results in TABLE 5 show that all spiked samples, containing viral genome copies of 240, 24, or 2.4, assayed using the TG-N2 assay, had a Cq value less than 40, while the negative PCR controls (NTC) appropriately had no amplification, i.e., no Cq value. With respect to the CoV-TGS01 assay, all spiked samples, containing viral genome copies of 240, 24, or 2.4, assayed using the CoV-TGS01 assay, had a Cq value less than 40, while the negative PCR controls (NTC) appropriately had no amplification, i.e., no Cq value. With the controls performing as expected, and the amplification curves for both TG-N2 and CoV-TGS01 crossing the threshold before 40 cycles, this indicates a positive test result for SAR-CoV-2 using both assays, TG-N2 and CoV-TGS01.

e. Analytical Sensitivity

The Limit of Detection (LoD), also called the Detection Limit or Lower Limit of Detection, is the lowest quantity of a substance that can be distinguished from the absence of that substance (i.e., a blank value) within a stated confidence limit. LoD is used to describe the sensitivity of quantitative assays.

The LoD was determined for the TG-N2 SARS-CoV-2 rRT-PCR Assay primer and probe set by limiting dilution studies using a stock of genomic RNA from the SARS-CoV-2 strain SARS-Related Coronavirus 2, Isolate USA-WA1/2020 (BEI Resources NR-52285) spiked into SARS-CoV-2-negative nasopharyngeal samples. The concentration in viral genome equivalents/mL in the RNA stock, 4.8×107 genome equivalents/mL, was supplied by BEI Resources and quantified by droplet digital PCR on a Biorad QX200.

For the TG-N2 primer and probe set, nine 1:2 serial dilutions of viral RNA spiked into real clinical matrix specimens starting from a concentration of 5×104 genome equivalents/mL of the characterized viral RNA were tested in three replicates. The lowest concentration at which all three replicates were positive was treated as the tentative LoD for each test. The LoD was then confirmed by testing concentrations at 1× the tentative LoD and 2× the tentative limit of detection with 20 replicates each. The final LoD of the TG-N2 primer/probe set was determined to be the lowest concentration resulting in positive detection of 19 out of 20 replicates.

TABLE 6 shows a summary of results for the TG-N2 assay 3-replicate limit of detection evaluation. Results show the LoD for TG-N2 is 1.56 genome equivalents/μL.

TABLE 6
Results for SARS-CoV-2 rRT-PCR Assay 3-replicate LoD evaluation

| | TG-N2 | CDC-RNase P |
|---|---|---|
| Genome | (mean (SD, % CV) | (mean (SD, % CV) |
| equivalents/μl | [N Amplified]) | [N Amplified]) |
| 50 | 33.65 (1.39, 4.1%) [3] | 23.89 (0.23, 1%) [3] |
| 25 | 34.7 (1.24, 3.6%) [3] | 23.96 (0.32, 1.3%) [3] |
| 12.5 | 35.6 (1.02, 2.9%) [3] | 24 (0.2, 0.8%) [3] |
| 6.25 | 36.33 (1.84, 5.1%) [3] | 23.91 (0.35, 1.5%) [3] |
| 3.13 | 37.15 (1.09, 2.9%) [3] | 24.15 (0.17, 0.7%) [3] |
| 1.56 | 38.46 (1.79, 4.7%) [3] | 24.01 (0.31, 1.3%) [3] |
| 0.78 | 39.65 (2.14, 5.4%) [2] | 23.72 (0.37, 1.6%) [3] |
| 0.39 | 40.74 (1.85, 4.5%) [2] | 23.5 (0.02, 0.1%) [3] |
| 0.2 | ND | 23.68 (0.39, 1.6%) [3] |

ND = Not detected;
SD = Standard Deviation;
CV = Coefficient of Variation;
N Amplified = Number of Replicates Amplified

Based on the results of the 3-replicate LoD evaluation, shown in TABLE 6, twenty (20) replicates each of sample spiked at 3.13, 1.56, 0.78, and 0.39 genome equivalents/μL were tested. Results of screening the TG-N2 primer and probe set on the lowest concentration at which 19 of 20 replicates amplified is shown in TABLE 7.

TABLE 7
Results of TG-N2 Assay 20-replicate LoD
evaluation

| Replicate | TG-N2 Assay | CDC-RNase P |
|---|---|---|
| number | Ct for 1.56 GEqs/μL | Ct |
| 1 | 37.05 | 23.18 |
| 2 | 37.16 | 23.26 |
| 3 | 39.3 | 23.22 |
| 4 | 38.04 | 23.22 |
| 5 | 38.11 | 23.52 |
| 6 | 36.95 | 23.41 |
| 7 | 36.51 | 23.17 |
| 8 | ND | 23.33 |
| 9 | 38.73 | 23.04 |
| 10 | 36.86 | 23.26 |
| 11 | 36.36 | 23.16 |
| 12 | 37.43 | 23.29 |
| 13 | 36.94 | 22.76 |
| 14 | 37.62 | 23.27 |
| 15 | 37.48 | 23.14 |
| 16 | 37.77 | 23.2 |
| 17 | 37.11 | 23.23 |
| 18 | 37.69 | 23.21 |
| 19 | 36.36 | 23.01 |
| 20 | 37.86 | 23.28 |

ND = Not detected

To summarize the results in TABLE 7 for the TG-N2 Assay, the mean Ct value was 37.4 with a standard deviation of 0.77 and a coefficient of variation of 2.0% for the 19 replicates detected, while the internal control (CDC-RNase P) had a mean Ct value of 23.2 with a standard deviation of 0.16 and a coefficient of variation of 0.67% for the 20 replicates detected.

f. Analytical Sensitivity (In Silico Inclusivity)

Inclusivity was assessed by nucleotide BLAST analysis of the National Center for Biotechnology Information (NCBI) nucleotide database (accessed 03/06/2020), with “SARS2 (taxid:2697049)” as the Organism filter, using each SARS-CoV-2 rRT-PCR Assay component as a query. All 48 SARS-CoV-2 complete genomes that were available at the time of database query (03/06/2020) hit at 100% identity to all primers and probes in the SARS-CoV-2 rRT-PCR Assay.

g. Analytical Specificity (in Silico Cross-Reactivity)

Each primer and probe was run through Basic Local Alignment Search Tool (BLAST®, National Center for Biotechnology Information, U.S. Laboratory of Medicine, Bethesda, Md.), to check for cross-reactivity to other relevant targets or species, excluding “SARS2 (taxid:2697049)” as the Organism filter, using default parameters for short input sequences, using each SARS-CoV-2 rRT-PCR Assay component as a query.

Cross-reactivity with organisms not in the NCBI database that may be present in a human nasopharyngeal sample was also assessed in silico. Shotgun metagenomic data of total RNA extractions from nine (9) nasopharyngeal samples were used as a query to identify any sequence reads that aligned using the Burrows Wheeler Aligner (BWA) to the primers and probes in the SARS-CoV-2 rRT-PCR Assay. Results showed that as a whole both the TG-N2 and CoV-TGS01 primer and probe sets are specific to SARS-CoV-2. Although individual primers and probe are not specific, none of the primer-probe sets hit the same organism-sequence in the NCBI nucleotide database, with two exceptions: accession no. MN996532.1, Bat coronavirus RaTG13, complete genome, and accession no. MT084071.1, Pangolin coronavirus isolate MP789 genomic sequence, which are both clinically irrelevant organisms.

### 2. Targeted Amplicon Sequencing

Amplicon-based sequencing can be used in the identification of one or more markers for the detection of SARS-CoV-2. Some embodiments of the invention include systems and methods of preparing samples for one or more downstream processes that can be used for assessing one or more markers for the detection of SARS-CoV-2.

For a targeted amplicon sequencing method, amplicon library preparation may be performed using the universal tail indexing strategy, i.e., using primers having universal tails. A universal indexing sequencing strategy can be used to amplify multiple genomic regions (e.g., markers, as described below) from a DNA sample simultaneously in a single reaction for the sequencing of one or more amplicons. Some embodiments of the invention comprise multiple steps and/or processes that are carried out to execute the universal tail indexing strategy to prepare amplicons for sequencing.

TABLE 8 shows primers for an amplicon sequencing assay targeting the N protein gene and the spike protein gene of SARS-CoV-2. In various embodiments, the SARS-CoV-2 amplicon sequencing assay comprises four amplicon sequencing assay primers (SEQ ID NOS: 9-12), and may further include indexing primers and sequencing primers.

TABLE 8

| | Assay | | | SEQ |
|---|---|---|---|---|
| | Component | Name | Sequence | ID NO: |
| N protein | forward | TG-N2_F | ACCCAACTGAATGGAGCTTC | 9 |
| gene: | primer | (AmpSeq) | AGCGTTCTTCGGAATGTC | |
| TG-N2 | reverse | TG-N2_R | ACGCACTTGACTTGTCTTCTG | 10 |
| assay | primer | (AmpSeq) | GCACCTGTGTAGGTCAAC | |
| S protein | forward | CoV-TS01_F | ACCCAACTGAATGGAGCGCA | 11 |
| gene: | primer | (AmpSeq) | CCTCATGGTGTAGTCTTCTTG | |
| CoV-TS01 | reverse | CoV-TS01_R | ACGCACTTGACTTGTCTTCTG | 12 |
| assay | primer | (AmpSeq) | GCAGGAGCAGTTGTGAA | |

| | | | |
|---|---|---|---|
| Universal Tails | UT1 | ACCCAACTGAATGGAGC | 13 |
| | UT2 | ACGCACTTGACTTGTCTTC | 14 |

An amplicon sequencing assay may include the TG-N2 (AmpSeq) primers, SEQ ID NOS: 9 and 10, and may further include the CoV-TS01 (AmpSeq) primers, SEQ ID NOS: 11 and 12.

In TABLE 8, the universal tails, which are added to the primers for amplicon sequencing, are underlined. Universal tail sequences are ACCCAACTGAATGGAGC (SEQ ID NO: 13) for forward read and ACGCACTTGACTTGTCTTC (SEQ ID NO: 14) for reverse read. The universal tail sequences (underlined) precede the assay-specific primer sequence (not underlined), for example, in SEQ ID NOS: 9, 10, 11 and 12.

The amplicon sequencing method may include creating a series of oligonucleotides designed to provide multiplexed amplification of one or more markers to produce the desired amplicons. After production of the amplicons (e.g., via PCR amplification), which may include the universal tail sequences, the resulting amplicons can be further processed to provide sequencing-ready amplicons. The method may further include performing downstream sequencing on the sequencing-ready amplicons.

The amplicon library preparation comprises two PCR steps, a gene-specific multiplex PCR and an index extension PCR.

First PCR: In gene-specific multiplex PCR reactions, the target amplicons are synthesized with a universal tail sequence added to the amplicons. Each primer includes a gene-specific sequence and a universal tail sequence, the universal tail sequences are underlined in TABLE 8. The forward primers have a first universal tail sequence, and the reverse primers have a second universal tail sequence, with the second universal tail sequence being different than the first universal tail sequence. For example, the forward primers (SEQ ID NOS: 9 and 11) include a first universal tail sequence (SEQ ID NO: 13), and the reverse primers (SEQ ID NOS: 10 and 12) include a second universal tail sequence (SEQ ID NO: 14). The amplification of the target results in the production of amplicons that comprise the first and second universal tail sequences integrated therein. After production of the amplicons during the multiplex PCR assay, the resulting amplicons can be further processed an indexing extension step to provide sequencing-ready amplicons.

Second PCR: The indexing extension PCR adds a specific index sequence to the amplicons using the universal tail sequences on either end of the amplicon. Stated differently, the amplicons are extended using platform-specific primers that recognize at least one of UT1 and UT2 for adding the indexes to each amplicon. The index is unique for each sample, such that the indexing primer includes a sample-specific index sequence and a common universal tail complement sequence. Thus, the number of different indexing primers used in the second PCR depends on the number of unique samples being processed in the same PCR. Each indexing primer comprises a complementary sequence that recognizes at least one of the first universal tail sequence and the second universal tail sequence that has been previously integrated within the amplicons. At the end of the index extension PCR there is a sequencer-ready amplicon library. By adding sample specific index sequences to the amplicons, pools of several samples are made ready for sequencing. The samples can be pooled for sequencing using a desired platform during a single sequencing run and distinguished based on the index sequence during analysis of the data. The inclusion of the universal tail sequences (SEQ ID NOS: 13 and 14) on the index and common primers may coincide with the use of genomic and index read primers in the mixture of sequencing primer reagents. After sequencing, the resulting data can be de-multiplexed and the sequence files can be aligned to a reference sequence (e.g., a wild type sequence and/or other alleles for each of the respective markers) for subsequent sequence analyses. As a result, the aligned sequences can be analyzed for the presence or absence of markers, variant signatures associated with the markers, differential marker presence in the sample, which includes the capability of analyzing gene expression, and an estimate of allele frequencies of various alleles of the markers in the pooled samples.

For example, the second PCR, using the universal tail-specific primers, adds Illumina's sample-specific index and sequencing adapters. Samples may then be pooled in equimolar concentration for sequencing. The amplicons may be sequenced by next-generation sequencing using a desired platform, such as the Illumina® MiSeq platform. Methods of sequencing include but need not be limited to any form of DNA sequencing including Sanger, next-generation sequencing, pyrosequencing, SOLiD sequencing, massively parallel sequencing, pooled, and barcoded DNA sequencing or any other sequencing method now known or yet to be disclosed. The number or quantity of sequencing reads for a particular gene or marker can be counted for each sample. In some aspects, the amplicons resulting from the multiplex PCR reaction can be sequenced, and the resulting sequences can be aligned to a reference sequence. As a result, differential numbers of sequence reads generated by the sequencing process (i.e., when aligned to the amplicon reference sequences), can provide data regarding the different copy numbers in the original RNA sample. The sequencing data or sequencing reads can be analyzed for identification and detection of SARS-CoV-2.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. An oligonucleotide having a 5′ terminus and a 3′ terminus, wherein the nucleotide sequence of the oligonucleotide consists of 42 or less nucleotides and has a nucleotide sequence that consists essentially of, or is a variant of, the nucleotide sequence of: SEQ ID NO:1, SEQ ID NO:2, SEQ ID NO:3, SEQ ID NO:5, SEQ ID NO:6, or SEQ ID NO:7.

2. The oligonucleotide of claim 1, wherein the variant thereof has no more than 5 substitutions, deletions, or additions.

3. The oligonucleotide of claim 1, wherein the oligonucleotide is modified with an internal spacer or a detectable label.

4. The oligonucleotide of claim 1, wherein the nucleotide sequence of the oligonucleotide further comprises a universal tail sequence.

5. (canceled)

6. The oligonucleotide of claim 1, wherein the nucleotide sequence of the oligonucleotide comprises SEQ ID NO:3 or SEQ ID NO:7.

7. The oligonucleotide of claim 1, wherein the 5′ terminus is labeled with a fluorophore and the 3′ terminus is complexed to a quencher of fluorescence of said fluorophore.

8. (canceled)

9. A kit for the detection of severe acute respiratory syndrome coronavirus 2 (SARS-CoV-2) in a biological sample comprising:
a primer pair, wherein at least one primer of the primer pair consists of less than 42 nucleotides and has a nucleotide sequence that consists essentially of, or is a variant of, the nucleotide sequence of: SEQ ID NO:1, SEQ ID NO:2, SEQ ID NO:5, or SEQ ID NO:6, and wherein the primer pair is capable of detecting SARS-CoV-2, if present, in the sample by amplification; and
SARS-CoV-2 detection reagents.

10. (canceled)

11. The kit of claim 9, wherein the variant thereof has no more than 5 substitutions, deletions, or additions.

12. The kit of claim 9, further comprising a probe modified with an internal spacer or detectable label, wherein the probe hybridizes to an oligonucleotide having a nucleotide sequence that consists essentially of SEQ ID NO:4 or SEQ ID NO:8.

13. (canceled)

14. The kit of claim 9, wherein the primer pair consists of: SEQ ID NO:1 and SEQ ID NO:2; or SEQ ID NO:5 and SEQ ID NO:6.

15-17. (canceled)

18. A method of detecting the presences of severe acute respiratory syndrome coronavirus 2 (SARS-CoV-2) polynucleotides in a biological sample in vitro, comprising:
(a) mixing the biological sample in vitro with a primer pair that is capable of amplifying a SARS-CoV-2 amplicon product, if the SARS-CoV-2 polynucleotide is present in the biological sample, wherein at least one primer of the primer pair consists of 42 or less nucleotides and has a nucleotide sequence that consists essentially of, or is a variant of, the nucleotide sequence of: SEQ ID NO:1, SEQ ID NO:2, SEQ ID NO:5, or SEQ ID NO:6;
(b) amplifying the SARS-CoV-2 amplicon product;
(c) contacting the SARS-CoV-2 amplicon product with a probe having a nucleotide sequence capable of hybridizing to the SARS-CoV-2 amplicon product, the probe being modified with an internal spacer or detectable label; and
(d) detecting whether SARS-CoV-2 polynucleotides are present in the biological sample by detecting the detectable label when the probe hybridizes to the SARS-CoV-2 amplicon.

19. (canceled)

20. The method of claim 18, wherein the primer pair comprises a first primer pair that amplifies a nucleocapsid protein amplicon product of SARS-CoV-2 and a second primer pair that amplifies a spike protein gene amplicon product of SARS-CoV-2.

21. The method of claim 18, wherein the amplicon product has a nucleotide sequence that consists essentially of SEQ ID NO:4 or SEQ ID NO:8.

22-23. (canceled)

24. The method of claim 18, wherein the primer pair consists of: SEQ ID NO:1 and SEQ ID NO:2; or SEQ ID NO:5 and SEQ ID NO:6.

25. The method of claim 24, wherein the primer pair includes at least two primer pairs comprising SEQ ID NO:1 and SEQ ID NO:2; and SEQ ID NO:5 and SEQ ID NO:6.

26. (canceled)

27. The method of claim 18, wherein the nucleotide sequence of the probe comprises the sequence of SEQ ID NO:3 or SEQ ID NO:7.

28. The method of 27, wherein the nucleotide sequence of the probe consists essentially of the sequence of SEQ ID NO:3 or the sequence of SEQ ID NO:7.

29-30. (canceled)

31. The method of claim 18, wherein the biological sample comprises a nasopharyngeal swab sample.

32-33. (canceled)

34. The kit of claim 9, wherein the primer pair comprises a first primer pair that amplifies a nucleocapsid protein amplicon product of SARS-CoV-2 and a second primer pair that amplifies a spike protein gene amplicon product of SARS-CoV-2:
the sequence of one primer of the first primer pair consists essentially of:
SEQ ID NO:1,
SEQ ID NO:1 and a universal tail sequence, or
SEQ ID NO:9;
the sequence of the other primer of the first primer pair consists essentially of:
SEQ ID NO:2,
SEQ ID NO:2 and a universal tail sequence, or
SEQ ID NO:10;
the sequence of one primer of the second primer pair consists essentially of:
SEQ ID NO:5,
SEQ ID NO:5 and a universal tail sequence, or
SEQ ID NO:11; and
the sequence of the other primer of the second primer pair consists essentially of:
SEQ ID NO:6,
SEQ ID NO:6 and a universal tail sequence, or
SEQ ID NO:12.

35-37. (canceled)

38. The method of claim 20, wherein:
the sequence of one primer of the first primer pair consists essentially of:
SEQ ID NO:1,
SEQ ID NO:1 and a universal tail sequence, or
SEQ ID NO:9;
the sequence of the other primer of the first primer pair consists essentially of:
SEQ ID NO:2,
SEQ ID NO:2 and a universal tail sequence, or
SEQ ID NO:10;
the sequence of one primer of the second primer pair consists essentially of:
SEQ ID NO:5,
SEQ ID NO:5 and a universal tail sequence, or
SEQ ID NO:11; and
the sequence of the other primer of the second primer pair consists essentially of:
SEQ ID NO:6,
SEQ ID NO:6 and a universal tail sequence, or
SEQ ID NO:12.


SEQUENCE LISTING (14 sequences)

SEQ ID NO: 1 - DNA, 21 bp, Artificial Sequence
  Primer
ttcagcgttcttcggaatgtc

SEQ ID NO: 2 - DNA, 20 bp, Artificial Sequence
  Primer
tggcacctgtgtaggtcaac

SEQ ID NO: 3 - DNA, 23 bp, Artificial Sequence
  Probe
cgcattggcatggaagtcacacc

SEQ ID NO: 4 - DNA, 77 bp, Severe Acute Respiratory Syndrome Coronavirus 2
ttcagcgttcttcggaatgtcgcgcattggcatggaagtcacaccttcgggaacgtggttgacctacacaggtgcca

SEQ ID NO: 5 - DNA, 24 bp, Artificial Sequence
  Primer
gcacctcatggtgtagtcttcttg

SEQ ID NO: 6 - DNA, 19 bp, Artificial Sequence
  Primer
tggcaggagcagttgtgaa

SEQ ID NO: 7 - DNA, 27 bp, Artificial Sequence
  Probe
catgtgacttatgtccctgcacaagaa

SEQ ID NO: 8 - DNA, 76 bp, Severe Acute Respiratory Syndrome Coronavirus 2
gcacctcatggtgtagtcttcttgcatgtgacttatgtccctgcacaagaaaagaacttcacaactgctcctgcca

SEQ ID NO: 9 - DNA, 38 bp, Artificial Sequence
  Primer
acccaactgaatggagcttcagcgttcttcggaatgtc

SEQ ID NO: 10 - DNA, 39 bp, Artificial Sequence
  Primer
acgcacttgacttgtcttctggcacctgtgtaggtcaac

SEQ ID NO: 11 - DNA, 41 bp, Artificial Sequence
  Primer
acccaactgaatggagcgcacctcatggtgtagtcttcttg

SEQ ID NO: 12 - DNA, 38 bp, Artificial Sequence
  Primer
acgcacttgacttgtcttctggcaggagcagttgtgaa

SEQ ID NO: 13 - DNA, 17 bp, Artificial Sequence
  Universal Tail
acccaactgaatggagc

SEQ ID NO: 14 - DNA, 19 bp, Artificial Sequence
  Universal Tail
acgcacttgacttgtcttc